(19) United States
(12) Patent Application Publication
(10) Pub. No.: US 2025/0318881 A1
(43) Pub. Date: Oct. 16, 2025

(54) SYSTEM AND METHOD FOR TRACKING OPHTHALMIC SURGICAL PROCEDURES

(71) Applicant: Alcon Inc., Fribourg (CH)

(72) Inventors: Lu Yin, Keller, TX (US); Vignesh Suresh, Woodland Hills, CA (US); Joseph Richard Weatherbee, San Clemente, CA (US); Ramesh Sarangapani, Coppell, TX (US)

(73) Assignee: Alcon Inc., Fribourg (CH)

(21) Appl. No.: 19/169,259

(22) Filed: Apr. 3, 2025

Related U.S. Application Data
(60) Provisional application No. 63/633,145, filed on Apr. 12, 2024.

(51) Int. Cl. A61B 34/20; A61B 3/10; A61B 3/14; A61F 9/007

(52) U.S. Cl. CPC A61B 34/20; A61B 3/102; A61B 3/14; A61F 9/00781; A61B 2034/2065

(57) ABSTRACT

A system for tracking an ophthalmic surgery. The system includes at least one digital camera configured to generate images of an eye, a memory comprising executable instructions, and an electronic control unit (ECU) in data communication with the memory. The ECU is configured to execute the executable instructions to collect images of an eye with at least a portion of the images being obtained intra-operatively and to create a spatial map of the eye based at least partially on the images of the eye. The ECU is also configured to execute the executable instructions to store at least one parameter regarding a clinical feature of the eye in connection with a spatial registration of clinical feature in the spatial map and recall a location of the clinical feature by tracking a location of the clinical feature in the spatial map.

### CROSS-REFERENCE TO RELATED APPLICATIONS

The present application claims the benefit of priority to U.S. Provisional Patent Application No. 63/633,145 filed on Apr. 12, 2024, which is hereby incorporated by reference in its entirety.

### TECHNICAL FIELD

The present disclosure relates generally to ophthalmic surgical procedures. More particularly, this disclosure relates to a system and method for tracking ophthalmic surgeries.

### BACKGROUND

Modern surgical procedures may employ a surgical microscope to provide a surgeon with a magnified view of target anatomy. Targeting magnification allows the surgeon to perform delicate surgical procedures on miniscule anatomical features or tissues. During a microscope-assisted procedure or microsurgery, magnified stereoscopic digital images of the target anatomy may be displayed within an operating suite via one or more high-resolution display screens, a heads-up display, or a set of oculars. Presentation of the magnified images in such a manner allows the surgeon to accurately visualize the target anatomy when evaluating its health or when maneuvering a tool in the performance of a surgical task.

### SUMMARY

Disclosed herein is a system for tracking an ophthalmic surgery. The system includes at least one digital camera configured to generate images of an eye, a memory comprising executable instructions, and an electronic control unit (ECU) in data communication with the memory. The ECU is configured to execute the executable instructions to collect images of an eye with at least a portion of the images being obtained intra-operatively and to create a spatial map of the eye based at least partially on the images of the eye. The ECU is also configured to execute the executable instructions to store at least one parameter regarding a clinical feature of the eye in connection with a spatial registration of clinical feature in the spatial map and recall a location of the clinical feature by tracking a location of the clinical feature in the spatial map.

In one embodiment, tracking the location of the clinical feature in the spatial map includes determining coordinates of the spatial registration for the clinical feature in the spatial map.

In one embodiment, the ECU is configured to execute the executable instructions to navigate a medical instrument towards the clinical feature identified in the spatial map.

In one embodiment, the ECU is configured to execute the executable instructions to navigate the medical instrument towards the clinical feature in the spatial map by generating an indicia of a location of the clinical feature relative to the medical instrument in the spatial map.

In one embodiment, the ECU is configured to execute the executable instructions to annotate the clinical feature in the spatial map.

In one embodiment, the ECU is configured to execute the executable instructions to scale the spatial map by comparing a dimension of a scaling feature in one of the plurality of images with the scaling feature in a pre-operatively obtained image with a known dimension.

In one embodiment, the scaling feature includes a limbus of the eye.

In one embodiment, the ECU is configured to execute the executable instructions to track the spatial registration of a cornea using patterns of a sclera and an iris of the eye scaled relative to a preoperatively obtained measurement of a diameter of a limbus of the eye.

In one embodiment, the ECU is configured to execute the executable instructions to utilize an image registration algorithm to generate an intra-operatively obtained spatial map of the eye having a field of view greater than a field of view of individual images of the plurality images.

In one embodiment, the spatial map includes a three-dimensional representation of at least a portion of the eye.

In one embodiment, the clinical feature includes an axis of the eye and tracking the location of the clinical feature includes indicating a location of the axis in the spatial map.

In one embodiment, the clinical feature includes a stent and the ECU is configured to execute the executable instructions to project a digital marker onto a sclera in the spatial map for assessing an efficiency of the stent in improving angle flow.

Disclosed herein is a method for tracking an ophthalmic surgery. The method includes collecting images of an eye with at least a portion of the images being obtained intra-operatively with a digital camera. A spatial map of the eye is created based on the images. At least one parameter is stored regarding a clinical feature of the eye in connection with a spatial registration of clinical feature in the spatial map and a location of the clinical feature is recalled by tracking a location of the clinical feature in the spatial map.

In one embodiment, tracking the location of the clinical feature in the spatial map includes determining coordinates for the clinical feature in the spatial map.

In one embodiment, navigating a medical instrument towards the clinical feature identified in the spatial map and generating an indicia of a location of the clinical feature relative to the medical instrument in the spatial map.

In one embodiment, determining a scale for the spatial map by comparing a dimension of a scaling feature in one of the plurality of images with the scaling feature in a pre-operatively obtained image having a known dimension.

In one embodiment, projecting a digital marker onto a sclera in the spatial map for assessing an efficiency of a stent in improving angle flow and the clinical feature includes a stent.

Disclosed herein is a computer-readable storage medium on which is recorded instructions for enhancing a digital image of a patient's eye during an ophthalmic procedure, wherein execution of the instructions by a processor causes the processor to collect images of an eye with at least a portion of the images being obtained intra-operatively and to create a spatial map of the eye based at least partially on the images of the eye. The execution of the instructions also cause the processor to execute the executable instructions to store at least one parameter regarding a clinical feature of the eye in connection with a spatial registration of clinical feature in the spatial map and to recall a location of the clinical feature by tracking a location of the clinical feature in the spatial map.

In one embodiment, tracking the location of the clinical feature in the spatial map includes determining coordinates for the clinical feature in the spatial map.

In one embodiment, navigating a medical instrument towards the clinical feature identified in the spatial map and generating an indicia of a location of the clinical feature relative to the medical instrument in the spatial map.

The above summary is not intended to represent every possible embodiment or every aspect of the subject disclosure. Rather, the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a representative operating suite for performing ophthalmic surgical procedures.

FIG. 2 is a schematic illustration of an example system for performing an ophthalmic surgical procedure within the representative operating suite of FIG. 1.

FIG. 3 illustrates a flowchart of an example method of tracking ophthalmic surgical procedures.

Some embodiments of the present disclosure are now described, by way of example only, and with reference to the accompanying drawings. The same reference number represents the same element or the same type of element on all drawings

### DETAILED DESCRIPTION

Embodiments of the present disclosure are described herein. It is to be understood, however, that the disclosed embodiments are merely examples and other embodiments can take various and alternative forms. The Figures are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

Ophthalmic surgical procedures, such as cataract surgery, vitreo-retina surgery, or microinvasive glaucoma surgery (MIGS), are very demanding tasks with surgeons mentally tracking the key clinical features (e.g., pathological condition encountered, or treatment conducted) intra-operatively at different location in the eye. This allows the surgeons to locate the clinical features for proper treatment at a later time in the surgery. For cataract surgery, one of the clinical features can include limbal relaxing incisions (LRI), which surgeons may need to refine after intra-operative lens (IOL) implantation. For vitreo-retina surgery, one of the clinical features can include a retina break or hole, which surgeons may need to close by using laser coagulation later. For MIGS, one of the clinical features can include a site of a stent insertion, which surgeons may need to check for episcleral blanching in a sclera region adjacent to the stent insertion site.

One feature of this disclosure is to aid surgeons by generating a spatial map of the eye for storing and recalling clinical features of the eye to minimize the surgeon's effort and time in re-locating them during the procedure. This can be helpful when conditions allow for clinical features to be more readily visible at an initial point in the procedure but become more difficult to see at a later stage of the surgery. Additionally, the clinical features may be out of the current field of view of the surgeon or imaging system and require navigation to regain visualization. This disclosure can also aid surgeons in tracking the completion of treatment in all necessary areas of the eye.

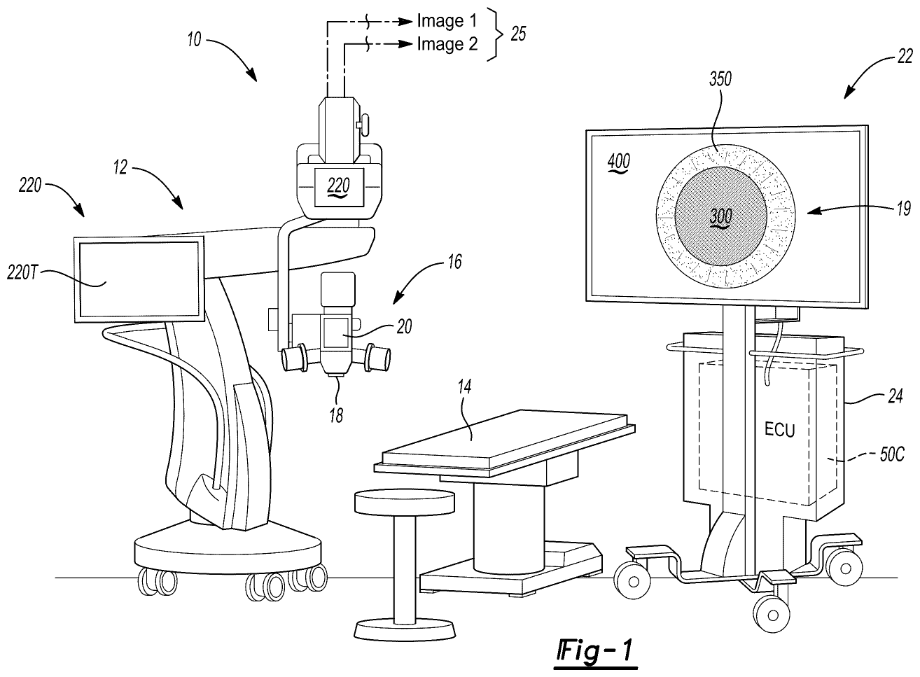

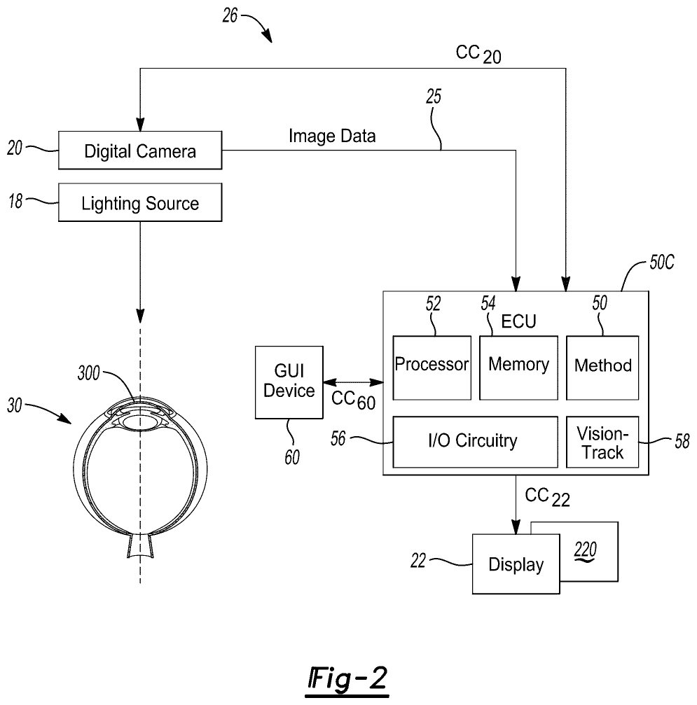

Referring now to the drawings wherein like reference numbers refer to like components, and beginning with FIG. 1, an operating suite 10 or system is depicted as it may appear during a representative eye surgery. As appreciated by those skilled in the art, the operating suite 10 may be equipped with a surgical robot 12 and an operating platform 14. The surgical robot 12 may be connected to a surgical microscope 16, e.g., a representative digital ophthalmic microscope, through which a surgeon (not shown) is able to view a patient's eye 30 (FIG. 2) or other target anatomy under application-suitable levels of magnification. A lighting source 18 and a digital camera 20 with one or more other image sensors may be coupled to or integral with the surgical microscope 16.

Using associated hardware and software of the surgical microscope 16 and an electronic control unit (ECU) 50C as described below, the surgeon is able to view images 19 of the target anatomy. Visualization may be facilitated via one or more high-resolution display screens 22 and/or 220, one or more of which may include a touch screen 220T, e.g., a capacitive display surface. As shown, the enhanced digital images 19 are of a target eye 30 of FIG. 2, with a representative image 19 in FIG. 1 including a pupil 300, a surrounding iris 350, and portions of the sclera 400.

Also present within the operating suite 10 is an optional cabinet 24 containing the ECU 50C, a processor 52 of which is shown in FIG. 2. The ECU 50C may be housed within the cabinet 24 in a possible implementation. Other embodiments are described below in which the processor 52 is integrated with or into other hardware within the operating suite 10 apart from the cabinet 24. Therefore, the illustrated implementation of FIG. 1 is non-limiting and exemplary, with the relevant processing functions of the ECU 50C and the processor(s) 52 described interchangeably below without regard to the particular location of either device.

The ECU 50C of FIG. 1 is configured herein to receive digital images (arrow 25), together forming a digital stereoscopic image as labeled “Image 1” and “Image 2” in FIG. 1. While collecting the digital images (arrow 25), the ECU 50C may execute computer-readable instructions embodying a method 50, an example of which is described below with reference to FIG. 2. The ECU 50C may be used as part of a system 26, representative hardware and software components of which are depicted in FIG. 1, with the system 26 (FIG. 2) in one or more implementations being operable for selectively enhancing the digital images (arrow 25) via surgeon's input and/or autonomous functions of the ECU 50C in a region-specific manner as set forth below.

The ECU 50C depicted in FIG. 1 is programmed with instructions or computer- executable code embodying one or more algorithms when implementing the method 50. When performing the method 50, the ECU 50C may present digital images 19 via any or all of the display screens 22 and/or 220, which may be alternatively embodied as oculars, binoculars, or heads-up displays (HUDs). That is, the contemplated digital image processing functions are performed by the ECU 50C in real-time, and in an unobtrusive and transparent manner from the perspective of the surgeon, so that the digital images 19 ultimately have desired region-specific spatial map of the eye 30.

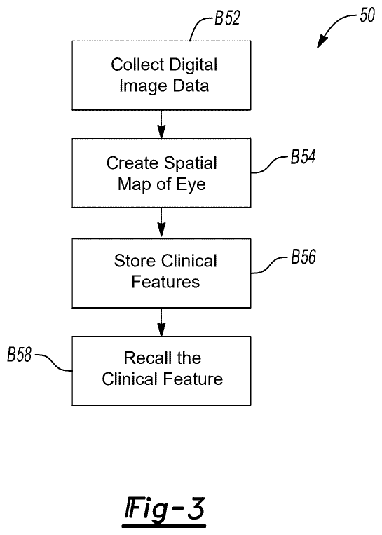

The system 26 of FIG. 2 in the illustrated exemplary embodiment also includes the above noted digital camera 20 but could use other types of imaging systems for performing ophthalmic surgical procedures. The digital camera 20 is operable for collecting digital images (arrow 25) as pixel image data of the patient's eye 30 under surgeon-selectable and/or procedure-specific illumination conditions. In an exemplary embodiment, the digital camera 20 may include a high-dynamic range (HDR) digital camera of the above-noted surgical microscope 16 of FIG. 1. Thus, components of the system 26 may be integral with the surgical microscope 16, i.e., an assembled internal or attached external component thereof, with the method 50 of FIG. 3 being programmed functionality of the surgical microscope 16.

Other embodiments may be realized in which instructions embodying the method 50 are recorded on a non-transitory computer-readable storage medium, e.g., in memory 54 of the ECU 50C, and executed by the processor(s) 52 of the ECU 50C as shown, or one or more processors 52 located apart from the ECU 50C in other embodiments with the memory 54 in data communication with the ECU 50C. Such structure would allow the ECU 50C to cause disclosed actions of the system 26 to occur. As noted above, the processor(s) 52 in alternative embodiments may be integrated into other hardware, e.g., the surgical microscope 16 and/or the digital camera 20, with inclusion of the processor(s) 52 in the construction of the ECU 50C being non-limiting.

The ECU 50C may command the digital camera 20, e.g., via corresponding camera control signals (arrow CC₂₀), to collect the digital images (arrow 25). The collected digital images (arrow 25) may be communicated or transmitted over transfer conductors and/or wirelessly to the processor(s) 52 for execution of the various digital image processing steps embodying the method 50. When selectively receiving the digital images (arrow 25), the processor(s) 52 of FIG. 2 may output a video display control signals (arrow CC₂₂) to the display screen(s) 22 and/or 220 to thereby cause the display screen(s) 22 and/or 220 (“Display(s)”) to display a spatial map of the patient's eye 30 as will be discussed in greater detail below. At other times, the digital camera 20 may be used as needed to image the eye 30.

The ECU 50C is depicted schematically in FIG. 2 as a unitary box solely for illustrative clarity and simplicity. Implemented embodiments of the ECU 50C may include one or more networked computer devices each with the processor(s) 52 and sufficient amounts of memory 54, the latter including a non-transitory (e.g., tangible) computer-readable storage medium on which is recorded or stored a set of computer-readable instructions, with such instructions embodying the functions of the method 50 being readable and executable by the processor(s) 52. An optional graphical user interface (GUI) device 60 may be used to facilitate intuitive interactions of the surgeon and attending surgical team with the system 26 via electronic output signals (CC₆₀) to the ECU 50C, with the electronic output signals (CC₆₀) being representative of the surgeon's inputs to the GUI device 60.

The memory 54 may take many forms, including but not limited to non-volatile media and volatile media. Instructions embodying the method 50 may be stored in the memory 54 and selectively executed by the processor(s) 52 to perform the various functions described below. The ECU 50C, either as a standalone device or integrated into the digital camera 20 and/or the surgical microscope 16 of FIG. 1, may also include resident machine vision/motion tracking logic 58 (“Vision-Track”) for tracking movement of the eye 30 during the microsurgery, and possibly performing other tasks like identifying a surgical tool, which may occur during the course of eye surgery as set forth below.

As will be appreciated by those skilled in the art, non-volatile computer readable storage media may include optical and/or magnetic disks or other persistent memory, while volatile media may include dynamic random-access memory (DRAM), static RAM (SRAM), etc., any or all which may constitute part of the memory 54 of the ECU 50C. The input/output (I/O) circuitry 56 may be used to facilitate connection to and communication with various peripheral devices used during the surgery, inclusive of the digital camera 20, the modulable lighting source 18, and the high-resolution display screen(s) 22 and/or 220. Other hardware not depicted but commonly used in the art may be included as part of the ECU 50C, including but not limited to a local oscillator or high-speed clock, signal buffers, filters, amplifiers, etc.

FIG. 3 schematically illustrates a flowchart for the example method 50 of tracking ophthalmic surgical procedures. The method 50 includes collecting intra-operatively obtained digital image data of the eye 30 at Block B52. In the illustrated example, the digital image data is obtained by an imaging system, such as the digital camera 20 shown in FIGS. 1-2 and can include the digital images 19. In one example, the digital images 19 include a field of view that at least partially overlaps with another one of the digital images 19.

With the intra-operatively obtained digital image data of the eye 30, the method 50 proceeds to Block B54 to create a spatial map of the eye 30. The spatial map can include a real time view of the eye 30 with a larger field of view than the digital camera 20 can capture in a single digital image 19. This allows the surgeon to view clinical features outside of the field of view of the digital images 19 being captured by the digital camera 20. This can reduce the burden on the surgeon to keep track of the clinical features that are outside of the field of view of the digital camera 20 and improve surgical planning by maintaining a record of the clinical features and the treatment performed at each region of the eye 30. The clinical feature may be tracked by determining a location of clinical feature with a spatial registration in a coordinate system describing the spatial map, such as a three-dimensional coordinate system.

In one example, the spatial map of the eye 30 is created or generated entirely from the intra-operatively obtained digital images 19. In another example, the spatial map is created by a combination of the intra-operatively obtained digital images 19 and pre-operatively obtained digital images of the eye 30. In one example, the method 50 creates the spatial map of the eye 30 by utilizing an image registration algorithm that gradually builds or stitches together individual intra-operatively digital images 19. Image registration may be used as described in U.S. patent application Ser. No. 18/299,029 to Yin et al., now published as US Patent Application Publication No. 2023/0334678A1, which was published on Oct. 19, 2023, and is hereby incorporated by reference in its entirety.

During certain surgical procedures, such as cataract surgery, the spatial map may include an anterior segment of the eye 30. This spatial map can be particularly beneficial during cataract surgery when implanting a toric intra-operative lens (IOL). When implanting the IOL, the surgeon aligns their operation on different axes of the patient eye 30 (e.g., astigmatism angle, visual axis, etc.). To create a scale for the spatial map of the eye 30 during cataract surgery, a location on the cornea within the patient's limbus is tracked. The cornea is tracked utilizing patterns of the sclera and the iris which are scaled according to the limbus diameter. The scaling of the spatial map is based on a comparison of a pre-operatively obtained measurement of the limbus diameter to an intra-operatively obtained measurement of the limbus diameter from the intra-operatively obtained digital images 19. However, features other than the limbus can be used as a scaling feature in the spatial map.

In another example surgical procedure, such as vitreoretinal surgery, a surgeon navigates to different regions of the patient eye 30 to operate (e.g., macular vs. periphery). Since a view generated by the digital camera 20 only encompasses a small portion of the fundus of the eye 30 at each moment, the surgeon must track all the details in the whole patient's eye 30, to plan the surgery. This allows the spatial map of the fundus (montaging) to include a field of view that is greater than the individual fields of view of fundus in the digital images 19.

In another example surgical procedure, such as MIGS surgery, a stent is implanted in the angle (e.g., at Trabecular meshwork). However, the efficacy of the flow through the stent is checked from an outside of the sclera. A location of the Gonioscopy view of the angle will be spatially aligned with the sclera image of the patient eye 30 as shown in the spatial map.

Once the spatial map has been created at Block B54, the spatial map and any parameters regarding the clinical feature(s) obtained during the creation of the spatial map are stored at Block B56 for later use, such as in the memory 54 of the ECU 50C in a transitory or non-transitory manner. The parameters regarding the clinical feature can include a location of the clinical feature in the spatial map that will allow the surgeon to return to view the clinical feature again during the surgery or to maintain a record of the clinical feature for tracking changes in the clinical feature over time.

One of the clinical features that may be stored during cataract surgery is a location of limbal relaxing incisions (LRI). Depending on the intra-op measurement of the eye 30, the surgeon may need to refine the initial LRI to better correct the residual astigmatism of the patient's eye 30. Due to the nature of wound healing of the cornea, the LRI made early in the procedure might be hard to see. The location and length of the LRI can be either automatically segmented through the use of an artificial intelligence (AI) algorithm or manually annotated by the surgeon or an assistant. This will allow the location of the LRI to be marked or visualized on the spatial map of the cornea and stored for later access. Another clinical feature that may be stored during cataract surgery can include edges of a rhexis within the eye 30.

Another clinical feature that may be identified during vitreoretinal surgery is a retinal break or hole. A shape of a retinal break or hole can either be automatically segmented through an AI algorithm as discussed above or manually annotated by the surgeon or the assistant. At time of storage, a location of the retinal break will be marked according to the spatial map of the fundus generated at Block B54. Additionally, as visualization can be a challenge for vitreo-retina surgery, visualization meta data such as the illumination intensity, image enhancement strategy will be recorded as well, to recreate the same optimized view at a later time in the procedure.

Regarding MIGS surgical procedures, one of the clinical features that can be stored is the insertion site of the stent, and how the stent location is mapped on the outside of the eye (sclera) from the angle. When a surgeon implants the stent at a specific site, the direction of the stent implantation can be either automatically detected by an AI algorithm or manually annotated by the surgeon or the assistant.

The method 50 can then proceed to Block B58 to determine if the clinical features that were previously identified in the spatial map and stored need to be recalled by the surgeon at a later point in the surgical procedure or during a post-operative follow-up appointment. The recalled clinical features can be visualized on the live view of the eye 30, such as from the spatial map. This allows for particular clinical features to be visualized by accounting for rotation of eye and magnification of the view. For surgical suites 10 with automation capability (e.g., motorized scopes, or robotic system), the location of the clinical features in the spatial map can be used to navigate the scope to the right field of view or control the movement of surgical instrument during the procedure or during a follow-up consultation to visualize healing of the eye 30 with the same or similar field of view stored in the spatial map of the clinical feature obtained during the surgical procedure.

Immediately after the surgery, the stored clinical features can be used to generate a check list to automate a billing process, as some of the treatment conducted by surgeons on those clinical features may include a complex procedure or additional billable events.

Furthermore, stored clinical features can function as intra-operatives notes to be ingested into electronic health records (EHR) or electronic medical record (EMR) systems. Stored clinical features, such as LRI, can be used as additional variables to improve the prediction of patient refractive outcomes. In the long term, stored clinical features, such as a retinal tear, can be used to track a recover status of the patient's eye 30, or to highlight potential risk to the patient at future diagnostics appointment or treatment planning.

Additionally, stored clinical features can be important variables on understanding how to improve the treatment efficacy of MIGS, and how the choice of insert sites and the extensiveness of episcleral blanching impact the long-term IOP level in patients.

For vitreoretinal surgery, recalling a location of the clinical features can occur by visualizing them on a large retina montage in the spatial map. Alternatively, a backend algorithm can align a current field of view of the digital camera 20 with the spatial map of the fundus and dynamically visualize clinical features stored for that field of view. In addition, by utilizing visualization meta data, visualization can be optimized for a live view generated by currently obtained digital images 19.

The spatial map can include indicia, such as arrows, which direct a medical instrument towards one of the clinical features when the surgeon wants to visualize the clinical feature at a later time in the surgical procedure. This is beneficial for vitreoretinal surgery, as the field of view of the scope typically only encompasses a small portion of the patient's fundus. Furthermore, there is also a scenario where the field of view of the scope or camera stays the same, but the clinical features become less visible during the treatment due to various reasons (e.g., hemorrhage). Presenting digital markers can aid the surgeon in consistently localizing the clinical features and directing the surgeon to a particular one of the clinical features.

For MIGS surgery, the location of clinical features in the angle can be visualized on the outside of the eye (sclera), taking the information of the site and direction of stent implantation, and physical dimension of the stent. A digital marker for the stent can be projected onto the sclera to aid the surgeon in assessing an efficacy of the stent in improving angle flow, by observing episcleral blanching in vessels next to the stent or applying anterior-segment optical coherence tomography (OCT) to image the angle and stent.

In general, storing and recalling clinical features of eye surgery can aid surgeons intra-operatively and in turn improve patient outcome. The method 50 can also help with different processes along the patient's journey, such as automatic billing, incorporating notes to EHR/EMR, and patient follow-up/appointment. The method 50 can interface with an inventory management system to determine a specific surgery type for purposes of generating billing codes and ordering supplies associated with the surgery type.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A system for tracking ophthalmic surgery, comprising:
at least one digital camera configured to generate a plurality images of an eye;
a memory comprising executable instructions; and
an electronic control unit (ECU) in data communication with the memory and configured to execute the executable instructions to:
collect a plurality of images of an eye, wherein at least a portion of the plurality of images are obtained intra-operatively;
create a spatial map of the eye based at least partially on the plurality of images of the eye;
store at least one parameter regarding a clinical feature of the eye in connection with a spatial registration of clinical feature in the spatial map; and
recall a location of the clinical feature by tracking a location of the clinical feature in the spatial map.

2. The system of claim 1, wherein tracking the location of the clinical feature in the spatial map includes determining coordinates of the spatial registration for the clinical feature in the spatial map.

3. The system of claim 2, wherein the ECU is configured to execute the executable instructions to navigate a medical instrument towards the clinical feature identified in the spatial map.

4. The system of claim 3, wherein the ECU is configured to execute the executable instructions to navigate the medical instrument towards the clinical feature in the spatial map by generating an indicia of a location of the clinical feature relative to the medical instrument in the spatial map.

5. The system of claim 1, wherein the ECU is configured to execute the executable instructions to annotate the clinical feature in the spatial map.

6. The system of claim 1, wherein the ECU is configured to execute the executable instructions to scale the spatial map by comparing a dimension of a scaling feature in one of the plurality of images with the scaling feature in a pre-operatively obtained image with a known dimension.

7. The system of claim 6, wherein the scaling feature includes a limbus of the eye.

8. The system of claim 7, wherein the ECU is configured to execute the executable instructions to track the spatial registration of a cornea using patterns of a sclera and an iris of the eye scaled relative to a preoperatively obtained measurement of a diameter of a limbus of the eye.

9. The system of claim 1, wherein the ECU is configured to execute the executable instructions to utilize an image registration algorithm to generate an intra-operatively obtained spatial map of the eye having a field of view greater than a field of view of individual images of the plurality images.

10. The system of claim 1, wherein the spatial map includes a three-dimensional representation of at least a portion of the eye.

11. The system of claim 1, wherein the clinical feature includes an axis of the eye and tracking the location of the clinical feature includes indicating a location of the axis in the spatial map.

12. The system of claim 1, wherein the clinical feature includes a stent and the ECU is configured to execute the executable instructions to project a digital marker onto a sclera in the spatial map for assessing an efficiency of the stent in improving angle flow by observing episcleral blanching in vessels next to the stent or applying anterior-segment optical coherence tomography (OCT) to image the angle and the stent.

13. A method for tracking an ophthalmic surgery, the method including:
collecting a plurality of images of an eye, wherein at least a portion of the plurality of images are obtained intra-operatively with a digital camera;
creating a spatial map of the eye based on the plurality of images;
storing at least one parameter regarding a clinical feature of the eye in connection with a spatial registration of clinical feature in the spatial map; and
recalling a location of the clinical feature by tracking a location of the clinical feature in the spatial map.

14. The method of claim 13, wherein tracking the location of the clinical feature in the spatial map includes determining coordinates for the clinical feature in the spatial map.

15. The method of claim 14, including navigating a medical instrument towards the clinical feature identified in the spatial map and generating an indicia of a location of the clinical feature relative to the medical instrument in the spatial map.

16. The method of claim 13, including determining a scale for the spatial map by comparing a dimension of a scaling feature in one of the plurality of images with the scaling feature in a pre-operatively obtained image having a known dimension.

17. The method of claim 13, including projecting a digital marker onto a sclera in the spatial map for assessing an efficiency of a stent in improving angle flow and the clinical feature includes a stent.

18. A computer-readable storage medium on which is recorded instructions for enhancing a digital image of a patient's eye during an ophthalmic procedure, wherein execution of the instructions by a processor causes the processor to:
collect a plurality of images of an eye, wherein at least a portion of the plurality of images are obtained intra-operatively with a digital camera;
create a spatial map of the eye based on the plurality of images;
store at least one parameter regarding a clinical feature of the eye in connection with a spatial registration of clinical feature in the spatial map; and
recall a location of the clinical feature by tracking a location of the clinical feature in the spatial map.

19. The computer-readable storage medium of claim 18, wherein tracking the location of the clinical feature in the spatial map includes determining coordinates for the clinical feature in the spatial map.

20. The computer-readable storage medium of claim 19, including navigating a medical instrument towards the clinical feature identified in the spatial map and generating an indicia of a location of the clinical feature relative to the medical instrument in the spatial map.
